SEQUENCE LISTING (14 sequences)

SEQ ID NO: 1 - DNA, 3162 bp, Staphylococcus aureus
  CDS (1)..(3162)
atgaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggctaa

SEQ ID NO: 2 - protein, 1053 aa, Staphylococcus aureus
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 3 - DNA, 3102 bp, Staphylococcus aureus
  CDS (1)..(3102)
atgaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaatccggcggcggttcgaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggctaa

SEQ ID NO: 4 - protein, 1033 aa, Staphylococcus aureus
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKSGGGSTEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 5 - DNA, 3072 bp, Staphylococcus aureus
  CDS (1)..(3072)
atgaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaatccggcggcggttcgaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggctaa

SEQ ID NO: 6 - protein, 1023 aa, Staphylococcus aureus
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKSGGGSTPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 7 - protein, 5 aa, Artificial Sequence
  Linker
SGGGS

SEQ ID NO: 8 - protein, 6 aa, Artificial Sequence
  Linker
GGSGGS

SEQ ID NO: 9 - protein, 8 aa, Artificial Sequence
  Linker
SGSGSGSG

SEQ ID NO: 10 - protein, 9 aa, Artificial Sequence
  Linker
SGSGSGSGS

SEQ ID NO: 11 - RNA, 20 nt, Artificial Sequence
  crRNA (C1)
acggaggcuaagcgucgcaa

SEQ ID NO: 12 - RNA, 21 nt, Artificial Sequence
  crRNA(A2)
ggagccacaguucuuccacgg

SEQ ID NO: 13 - RNA, 21 nt, Artificial Sequence
  crRNA(A3)
cucuacccuugaggucucgag

SEQ ID NO: 14 - RNA, 83 nt, Artificial Sequence
  tracrRNA
guuuuaguacucuggaaacagaaucuacuaaaacaaggcaaaaugccguguuuaucacgucaacuuguuggcgagauuuuuuu

(19) United States
(12) Patent Application Publication
(10) Pub. No.: US 2021/0246473 A1
(43) Pub. Date: Aug. 12, 2021

(54) MODIFIED CAS9 PROTEIN, AND USE THEREOF

(71) Applicant: MODALIS THERAPEUTICS CORPORATION, Chuo-ku (JP)

(72) Inventor: Yuanbo QIN, Cambridge, MA (US)

(73) Assignee: MODALIS THERAPEUTICS CORPORATION, Chuo-ku (JP)

(21) Appl. No.: 17/288,379

(22) PCT Filed: Oct. 24, 2019
(86) PCT No.: PCT/JP2019/041751

§ 371 Date: Apr. 23, 2021

Related U.S. Application Data
(60) Provisional application No. 62/749,855, filed on Oct. 24, 2018.

(51) Int. Cl. C12N 15/90; C12N 9/22; C12N 15/11

(52) U.S. Cl. CPC C12N 15/907; C12N 2310/20; C12N 15/11; C12N 9/22

(57) ABSTRACT

The present invention relates to a protein having a binding ability to guide RNA and consisting of a sequence comprising an amino acid sequence wherein a continuous deletion region is present between the 721-position and the 755-position in the amino acid sequence shown in SEQ ID NO: 2, wherein amino acids adjacent to each of the deletion region are linked by a linker consisting of 3 to 10 amino acid residues. The binding ability to the guide RNA, and further, the DNA binding affinity, are maintained even though the protein has a deletion region and is smaller than the full-length dSaCas9. Use of the miniaturized dSaCas9 protein makes it possible to mount many genes into vectors.

### TECHNICAL FIELD

The present invention relates to a modified Cas9 protein that is miniaturized while maintaining a binding ability to guide RNA, and use thereof.

### BACKGROUND ART

Clustered Regularly Interspaced Short Palindromic Repeats (CRISPR) are known to compose the adaptive immune system that provides acquired resistance against invasive foreign nucleic acids in bacteria and archaea together with Cas (CRISPR-associated) genes. CRISPR frequently originate from phage or plasmid DNA and are composed of 24 bp to 48 bp short, conserved repeat sequences having unique variable DNA sequences referred to as spacers of similar size inserted there between. In addition, a group of genes encoding the Cas protein family is present in the vicinity of the repeat and spacer sequences.

In the CRISPR/Cas system, foreign DNA is cleaved into fragments of about 30 bp by the Cas protein family and inserted into CRISPR. Cas1 and Cas2 proteins, which are among the Cas protein family, recognize a base sequence referred to as proto-spacer adjacent motif (PAM) of foreign DNA, cut the upstream, and insert same into the CRISPR sequence of the host, which creates immune memory of bacteria. RNA generated by transcription of a CRISPR sequence including immune memory (referred to as pre-crRNA) is paired with a partially complementary RNA (trans-activating crRNA: tracrRNA) and incorporated into Cas9 protein which is one of the Cas protein family. The pre-crRNA and tracrRNA incorporated into Cas9 are cleaved by RNaseIII to form small RNA fragments (CRISPR-RNAs: crRNAs) containing a foreign sequence (guide sequence), and a Cas9-crRNA-tracrRNA complex is thus formed. The Cas9-crRNA-tracrRNA complex binds to a foreign invasive DNA complementary to crRNA, and the Cas9 protein, which is an enzyme that cleaves the DNA (nuclease), cleaves the foreign invasive DNA, thereby suppressing and eliminating the function of the DNA that invaded from the outside.

In recent years, techniques for applying the CRISPR/Cas system to genome editing have been actively developed. crRNA and tracrRNA are fused, expressed as a tracrRNA-crRNA chimera (hereinafter to be referred to as guide RNA: gRNA), and utilized. Using this, nuclease (RNA-guided nuclease: RGN) is then recruited to cleave genomic DNA at the target site.

On the other hand, a system that can regulate the expression level of the target gene can be obtained by fusing a transcriptional regulator such as transcriptional activator (e.g., VP64, VP160 and the like), a transcriptional inhibitor (e.g., KRAB and the like), and the like with a variant (nuclease-null, dCas9) wherein the nuclease of the Cas9 protein in CRISPR/Cas9, which is one of the genome editing systems, is inactivated. For example, to further increase the efficiency of gene activation, it is fused with an activation factor (VP64-Rta) in which two transcriptional activators are linked or an activation factor (VP64-p65-Rta, VPR) in which three transcriptional activators are linked, and the fused dCas9 protein (dCas9-VPR; dCas9 fusion protein) strongly activates expression of the target gene without cleaving DNA.

Various variants of Cas9 protein have been created and reported for the purpose of alleviating PAM specificity, modifying (activating/inactivating) nuclease activity, and miniaturizing size (patent documents 1-3).

### DOCUMENT LIST

### Patent Documents

patent document 1: WO 2016/141224A1
patent document 2: WO 2017/010543A1
patent document 3: WO 2018/074979A1

### SUMMARY OF INVENTION

### Technical Problem

Expression of dCas9 fusion protein in vivo requires an expression vector. In gene therapy, adeno-associated virus vector (AAV) is mainly used since it is highly safe and highly efficient. The mountable size of AAV is about 4.4 kb, while the dCas9 protein already occupies about 4 kb, and the constituent of fusion protein is extremely limited for mounting on AAV.

Therefore, the present inventors aim to provide a further miniaturized dCas9 protein variant having DNA binding affinity substantially equivalent to that of a full-length protein.

### Solution to Problem

The present inventors took note of a nuclease-null variant (dSaCas9) of Cas9 derived from S. aureus (to be also referred to as SaCas9 in the present description) as a Cas9 protein, and have conducted intensive studies in an attempt to solve the above-mentioned problems. As a result, they have found a specific region that has little influence on the ability to bind to guide RNA even if deleted, and succeeded in producing a miniaturized dSaCas9 protein while maintaining or enhancing the DNA binding affinity by substituting the amino acid at a predetermined position with a specific amino acid, which resulted in the completion of the present invention.

Deletion and substitution are also collectively referred to as mutation.

In the present description, dSaCas9 protein before introduction of mutation is sometimes to be referred to as wild-type dSaCas9 (protein), and dSaCas9 protein after introduction of mutation is sometimes to be referred to as modified dSaCas9 variant (protein).

That is, the present invention provides the following.

[1] A protein having a binding ability to guide RNA and consisting of a sequence comprising an amino acid sequence wherein a continuous deletion region is present between the 721-position and the 755-position in the amino acid sequence shown in SEQ ID NO: 2,
wherein amino acids adjacent to each of the deletion region are linked by a linker consisting of 3 to 10 amino acid residues.
[2] The protein of the above-mentioned [1], wherein the linker is a 5-9 amino acid length linker composed of glycine (G) and serine (S).
[3] The protein of the above-mentioned [2], wherein the linker is selected from the following:

### —SGGGS—

### —GGSGGS—

### —SGSGSGSG—

### —SGSGSGSGS—.

[4] The protein of any of the above-mentioned [1] to [3], wherein the deletion region is a region of the 721-position to the 745-position.
[5] The protein of the above-mentioned [4], wherein the protein is shown in SEQ ID NO: 4.
[6] The protein of any of the above-mentioned [1] to [3], wherein the deletion region is a region of the 721-position to the 755-position.
[7] The protein of the above-mentioned [6], wherein the protein is shown in SEQ ID NO: 6.
[8] The protein of any of the above-mentioned [1] to [7], wherein glutamic acid (E) at the 45-position and/or the 163-position are/is substituted with other amino acid(s).
[9] The protein of the above-mentioned [8], wherein said other amino acid is a basic amino acid.
[10] The protein of the above-mentioned [9], wherein the basic amino acid is lysine (K).
[11] The protein of any of the above-mentioned [1] to [10], having identity of 80% or more at a site other than the mutated and/or deleted positions in the SEQ ID NO: 2.
[12] The protein of any of the above-mentioned [1] to [10], wherein one to several amino acids are substituted, deleted, inserted and/or added at a site other than the mutated and/or deleted positions in the SEQ ID NO: 2.
[13] The protein of any of the above-mentioned [1] to [12], wherein a transcriptional regulator protein or domain is linked.
[14] The protein of the above-mentioned [13], wherein the transcriptional regulator is a transcriptional activator.
[15] The protein of the above-mentioned [13], wherein the transcriptional regulator is a transcriptional silencer or a transcriptional inhibitor.
[16] A nucleic acid encoding the protein of any of the above-mentioned [1] to [15].
[17] A protein-RNA complex provided with the protein of any of the above-mentioned [1] to [16] and a guide RNA comprising a polynucleotide composed of a base sequence complementary to a base sequence located 1 to 20 to 24 bases upstream from a proto-spacer adjacent motif (PAM) sequence in a target double-stranded polynucleotide.
[18] A method for site-specifically modifying a target double-stranded polynucleotide, including

a step of mixing and incubating a target double-stranded polynucleotide, a protein and a guide RNA, and

a step of having the aforementioned protein modify the aforementioned target double-stranded polynucleotide at a binding site located upstream of a PAM sequence; wherein,

the aforementioned protein is the protein of any of the above-mentioned [1] to [15], and

the aforementioned guide RNA contains a polynucleotide composed of a base sequence complementary to a base sequence located 1 to 20 to 24 bases upstream from the aforementioned PAM sequence in the aforementioned target double-stranded polynucleotide.

[19] A method for increasing expression of a target gene in a cell, comprising expressing the protein of the above-mentioned [14] and one or plural guide RNAs for the aforementioned target gene in the aforementioned cell.
[20] A method for decreasing expression of a target gene in a cell, comprising expressing the protein of the above-mentioned [15] and one or plural guide RNAs for the aforementioned target gene in the aforementioned cell.
[21] The method of the above-mentioned [19] or [20], wherein the cell is a eukaryotic cell.
[22] The method of the above-mentioned [19] or [20], wherein the cell is a yeast cell, a plant cell or an animal cell.

### Advantageous Effects of Invention

According to the present invention, a dSaCas9 protein further miniaturized while having a binding ability to guide RNA can be obtained. The miniaturized dSaCas9 protein makes it possible to mount a larger number of genes into expression vectors limited in capacity.

### BRIEF DESCRIPTION OF DRAWINGS

FIG. 1 is a schematic showing of the structure of wild-type dSaCas9(FL) and dSaCas9 variants (T10-T11). Both ends of the deletion region (721-745 positions) in T10 and both ends of the deletion region (721-755 positions) in T11 are linked by a linker peptide (SGGGS).

FIG. 2 is a graph showing the results of suppression of the expression of the MyD88 gene by guide RNA using A2 (SEQ ID NO: 12) as crRNA and a molecule in which wild-type dSaCas9 (FL) or dSaCas9 variant (T10, T11) is linked to the KRAB gene.

FIG. 3 is a graph showing the results of suppression of the expression of the MyD88 gene by guide RNA using A2 (SEQ ID NO: 12) or A3 (SEQ ID NO: 13) as crRNA and a molecule in which wild-type dSaCas9 (FL) or dSaCas9 variant (T10) is linked to the KRAB gene.

### DESCRIPTION OF EMBODIMENTS

The present invention is described below. Unless particularly indicated, the terms used in the present description have meanings generally used in the pertinent field.

<dSaCas9 variant>

The dSaCas9 variant of the present invention is a dSaCas9 protein further miniaturized while having a binding ability to guide RNA. Using the miniaturized dSaCas9 protein, a larger number of genes can be mounted into a vector.

In the present description, “guide RNA” refers to that which mimics the hairpin structure of tracrRNA-crRNA, and contains in the 5′-terminal region thereof a polynucleotide composed of a base sequence complementary to a base sequence located from 1 to preferably 20 to 24 bases, and more preferably from 1 to preferably 22 to 24 bases, upstream from the PAM sequence in a target double-stranded polynucleotide. Moreover, guide RNA may contain one or more polynucleotides composed of a base sequence allowing the obtaining of a hairpin structure composed of base sequences non-complementary to a target double-stranded polynucleotide symmetrically arranged so as to form a complementary sequence having a single point as the axis thereof.

The guide RNA has a function of binding to the dSaCas9 variant of the present invention and leading the protein to a target DNA. The guide RNA has a sequence at the 5′-terminal which is complementary to the target DNA, and binds to the target DNA via the complementary sequence, thereby leading the dSaCas9 variant of the present invention to the target DNA. Since the dSaCas9 variant does not have a DNA endonuclease, it does not cleave target DNA though it binds to the target DNA.

The guide RNA is designed and prepared based on the sequence information of the target DNA. Specific examples include sequences such as those used in the Examples.

In the present description, the terms “polypeptide”, “peptide” and “protein” refer to polymers of amino acid residues and are used interchangeably. In addition, these terms also refer to amino acid polymers in which one or a plurality of amino acid residues are in the form of a chemical analog or modified derivative of the corresponding amino acids present in nature.

In the present description, the “basic amino acid” refers to an amino acid having a residue showing basicity in addition to one amino group in a molecule such as lysine, arginine, histidine and the like.

In the present description, a “sequence” refers to a nucleotide sequence of an arbitrary length, is a deoxyribonucleotide or ribonucleotide, and may be linear or branched and single-stranded or double-stranded.

In the present description, a “PAM sequence” refers to a sequence present in a target double-stranded polynucleotide that can be recognized by Cas9 protein, and the length and base sequence of the PAM sequence differs according to the bacterial species.

Furthermore, in the present description, “N” refers to any one base selected from the group consisting of adenine, cytosine, thymine and guanine, “A” refers to adenine, “C” to guanine, “C” to cytosine, “T” to thymine, “R” to a base having a purine skeleton (adenine or guanine), and “Y” to a base having a pyrimidine skeleton (cytosine or thymine).

In the present description, a “polynucleotide” refers to a deoxyribonucleotide or ribonucleotide polymer having linear or cyclic coordination and may be single-stranded or double-stranded, and should not be interpreted as being restricted with respect to polymer length. In addition, polynucleotides include known analogs of naturally-occurring nucleotides as well as nucleotides in which at least one of the base moieties, sugar moieties and phosphate moieties thereof has been modified (such as a phosphorothioate backbone). In general, an analog of a specific nucleotide has the same base-pairing specificity, and for example, A analogs form base pairs with T.

The present invention provides a protein having a binding ability to guide RNA and consisting of a sequence comprising an amino acid sequence wherein a continuous deletion region is present between the 721-position and the 755-position in the amino acid sequence shown in SEQ ID NO: 2, wherein amino acids adjacent to each of the deletion region are linked by a linker consisting of 3 to 10 amino acid residues (embodiment 1).

SEQ ID NO: 2 is a full-length amino acid sequence of dSaCas9 protein. The dSaCas9 protein consists of two lobes of REC lobe (41-425 residues) and NUC lobe (1-40 residues and 435-1053 residues). The two lobes are linked via bridge helix (BH: 41-73 residues) rich in arginine and linker loop (426-434 residues). The NUC lobe is constituted of RuvC domain (1-40, 435-480 and 650-774 residues), HNH domain (520-628 residues), WED domain (788-909 residues) and PI domain (910-1053 residues). The PT domain is divided into topoisomerase homology (TOPO) domain and C-terminal domain (CTD). The RuvC domain is constituted of 3 separate motifs RuvC-I-III) and is associated with the HNH domain and PI domain. The HNH domain is linked to RuvC-II and RuvC-III via L1 (481-519 residues) linker and L2 (629-649 residues) linker, respectively. The WED domain and RucV domain are linked by “phosphate lock” loop (775-787 residues).

In one embodiment of the present invention, the continuous deletion region present between the 721-position and the 755-position in the amino acid sequence shown in SEQ ID NO: 2 is a region of the 721- to 745-positions (embodiment 1-1).

Examples of the protein of embodiment 1-1 and the gene encoding the same respectively include a protein consisting of the amino acid sequence shown in SEQ ID NO: 4 and a gene consisting of the base sequence shown in SEQ ID NO: 3. The protein corresponds to the dSaCas9 variant T10 described below.

In one embodiment of the present invention, the continuous deletion region present between the 721-position and the 755-position in the amino acid sequence shown in SEQ ID NO: 2 is a region of the 721- to 755-positions (embodiment 1-2).

Examples of the protein of embodiment 1-2 and the gene encoding the same respectively include a protein consisting of the amino acid sequence shown in SEQ ID NO: 6 and a gene consisting of the base sequence shown in SEQ ID NO: 5. The protein corresponds to the dSaCas9 variant T11 described below.

In another embodiment of the present invention, the present invention provides a protein (embodiment 2) having binding ability to guide RNA and further having mutations at the 45-position and/or the 163-position in addition to the mutations in the aforementioned embodiments 1, 1-1 and 1-2.

The mutation(s) at the 45-position and/or the 163-position are/is specifically substitution of glutamic acid with basic amino acid, preferably with lysine, arginine or histidine, more preferably with lysine.

As a method for optionally creating “a continuous deletion region between the 721-position and the 755-position” in the amino acid sequence shown in SEQ ID NO: 2, and a method for “substituting glutamic acid at the 45-position and/or the 163-position with other amino acid” in the amino acid sequence shown in SEQ ID NO: 2, a method including introducing a conventional site-specific mutation into a DNA encoding a predetermined amino acid sequence, and then expressing the DNA by a conventional method can be mentioned. Examples of the method for introducing a site-specific mutation include a method using amber mutation (gapped-duplex method, Nucleic Acids Res., 12, 9441-9456 (1984)), a method by PCR using a primer for mutagenesis, and the like. In addition, it can be easily performed according to the manual and using the Q5 Site-Directed Mutagenesis Kit (NEB).

In another embodiment of the present invention, the present invention provides a protein (embodiment 3) that is functionally equivalent to the proteins of the aforementioned embodiments 1, 1-1, 1-2 and 2. To be functionally equivalent to the proteins of the aforementioned embodiments 1, 1-1, 1-2 and 2, the amino acid sequence has identity of 80% or more at a site other than the positions where the mutations have been applied in the SEQ ID NO: 2 in the aforementioned embodiments 1, 1-1, 1-2 and 2 and has a binding ability to guide RNA. When amino acids are increased or decreased due to mutation, the “site other than the position(s) where the mutation(s) has(have) been applied” can be interpreted to mean a “site other than the position(s) corresponding to the position(s) where the mutation(s) has(have) been applied”. This identity is preferably 80% or more, more preferably 85% or more, even more preferably 90% or more, particularly preferably 95% or more, and most preferably 99% or more. The amino acid sequence identity can be determined by a method known per se. For example, amino acid sequence identity (%) can be determined using a program conventionally used in the pertinent field (e.g., BLAST, FASTA, etc.) by default. In another aspect, identity (%) is determined by any algorithm known in the pertinent field, such as algorithms of Needleman et al. (1970) (J. Mol. Biol. 48: 444-453), Myers and Miller (CABIOS, 1988, 4: 11-17) and the like. The algorithm of Needleman et al. is incorporated into the GAP program in the GCG software package (available at www.gcg.com) and the identity (%) can be determined using, for example, any of BLOSUM 62 matrix and PAM250 matrix, as well as gap weight: 16, 14, 12, 10, 8, 6 or 4, and length weight: 1, 2, 3, 4, 5 or 6. The algorithm of Myers and Miller is incorporated into the ALIGN program that is a part of the GCG sequence alignment software package. When the ALIGN program is used to compare amino acid sequences, for example, PAM120 weight residue table, gap length penalty 12, and gap penalty 4 can be used.

As a protein functionally equivalent to the proteins of the aforementioned embodiments 1, 1-1, 1-2 and 2, a protein (embodiment 3-1) which comprises one to several amino acids substituted, deleted, inserted and/or added at site(s) other than the positions where the mutations have been applied in the SEQ ID NO: 2 in the aforementioned embodiment 1, 1-1, 1-2 and 2 and having the binding ability to guide RNA is provided. When amino acids are increased or decreased due to mutation, the “site other than the position(s) where the mutation(s) has(have) been applied” can be interpreted to mean a “site other than the position(s) corresponding to the position(s) where the mutation(s) has(have) been applied”.

As a technique for artificially performing “substitution, deletion, insertion and/or addition of amino acid”, for example, a method including applying conventional site specific mutation introduction to DNA encoding a predetermined amino acid sequence, and thereafter expressing the DNA by a conventional method can be mentioned. Examples of the site specific mutation introduction method include a method using amber mutation (gapped duplex method, Nucleic Acids Res., 12, 9441-9456 (1984)), a PCR method using a mutation introduction primer and the like. In addition, it can be easily performed according to the manual and using the Q5 Site-Directed Mutagenesis Kit (NEB).

The number of the amino acids modified above is at least one residue, specifically one or several, or more than that. Among the aforementioned substitution, deletion, insertion and addition, substitution of amino acid is particularly preferred. The substitution is more preferably substitution with an amino acid having similar properties such as hydrophobicity, charge, pK, and characteristic of steric structure and the like. Examples of the substitution include substitution within the groups of i) glycine, alanine; ii) valine, isoleucine, leucine; iii) aspartic acid, glutamic acid, asparagine, glutamine; iv) serine, threonine; v) lysine, arginine; vi) phenylalanine, tyrosine.

In the dSaCas9 variant of the present invention, the dSaCas9 variant protein is in a state of being cleaved by the deletion mutation, and the both ends of the deletion region are linked by a linker. That is, in the dSaCas9 variant of the present invention, amino acids each adjacent to the deletion region are linked by a linker consisting of 3 to 10 amino acid residues. Due to the linkage, the dSaCas9 variant of the present invention has a continuous amino acid sequence.

The linker is not particularly limited as long as it can link both ends of a cleaved protein and does not influence the function thereof. Preferably, it is a group capable of adopting an intrinsically disordered structure that binds to other protein while freely changing its own shape according to the protein, and more preferably a peptide residue having a length of 5-9 amino acids which is constituted of glycine (G) and serine (S). Specifically, the following residues can be mentioned.

(SEQ ID NO: 7)
-SGGGS-
(SEQ ID NO: 8)
-GGSGGS-
(SEQ ID NO: 9)
-SGSGSGSG-
(SEQ ID NO: 10)
-SGSGSGSGS-

In the dSaCas9 variants T10 and T11, -SGGGS- is used as the linker.

The introduction of linker in each variant can also be performed by a method including performing conventional site-specific mutagenesis on the DNA encoding a predetermined amino acid sequence to insert a base sequence encoding the linker, and thereafter expressing the DNA by a conventional method. Examples of the method for site-specific mutagenesis include methods same as those described above.

The dSaCas9 variant in the present embodiment can be produced according to, for example, the method indicated below. First, a host is transformed using a vector containing a nucleic acid that encodes the dSaCas9 variant of the present invention. The n, the host is cultured to express the aforementioned protein. Conditions such as medium composition, culture temperature, duration of culturing or addition of inducing agents can be determined by a person with ordinary skill in the art in accordance with known methods so that the transformant grows and the aforementioned protein is efficiently produced. In addition, in the case of having incorporated a selection marker in the form of an antibiotic resistance gene in an expression vector, the transformant can be selected by adding antibiotic to the medium. The n, dSaCas9 variant of the present invention is obtained by purifying the aforementioned protein expressed by the host according to a method known per se.

There are no particular limitations on the host, and examples thereof include animal cells, plant cells, insect cells and microorganisms such as Escherichia coli, Bacillus subtilis or yeast. Preferred host is an animal cell.

<dSaCas9 Variant-Guide RNA Complex>

In one embodiment thereof, the present invention provides a protein-RNA complex provided with the protein indicated in the previous section on <dSaCas9 variant> and guide RNA containing a polynucleotide composed of a base sequence complementary to a base sequence located 1 to 20 to 24 bases upstream from a proto-spacer adjacent motif (PAM) sequence in a target double-stranded polynucleotide.

The aforementioned protein and the aforementioned guide RNA are able to form a protein-RNA complex by mixing in vitro and in vivo under mild conditions. Mild conditions refer to a temperature and pH of a degree that does not cause proteolysis or denaturation, and the temperature is preferably 4° C. to 40° C., while the pH is preferably 4 to 10.

In addition, the duration of mixing and incubating the aforementioned protein and the aforementioned guide RNA is preferably 0.5 hr to 1 hr. The complex formed by the aforementioned protein and the aforementioned guide RNA is stable and is able to maintain stability even if allowed to stand for several hours at room temperature.

### <CRISPR-Cas Vector System>

In one embodiment thereof, the present invention provides a CRISPR-Cas vector system provided with a first vector containing a gene encoding a protein indicated in the previous section on <dSaCas9 variant>, and a second vector containing a guide RNA containing a polynucleotide composed of a base sequence complementary to a base sequence located 1 to 20 to 24 bases upstream from PAM sequence in a target double-stranded polynucleotide.

In another embodiment, the present invention provides a CRISPR-Cas vector system in which a gene encoding a protein indicated in the previous section on <dSaCas9 variant>, and a guide RNA containing a polynucleotide composed of a base sequence complementary to a base sequence located 1 to 20 to 24 bases upstream from PAM sequence in a target double-stranded polynucleotide are contained in the same vector.

The guide RNA is suitably designed to contain in the 5′-terminal region thereof a polynucleotide composed of a base sequence complementary to a base sequence located from 1 to 20 to 24 bases, and preferably to 22 to 24 bases, upstream from a PAM sequence in a target double-stranded polynucleotide. oreover, the guide RNA may also contain one or more polynucleotides composed of a base sequence allowing the obtaining of a hairpin structure composed of base sequences non-complementary to a target double-stranded polynucleotide symmetrically arranged so as to form a complementary sequence having a single point as the axis thereof.

The vector of the present embodiment is preferably an expression vector. Examples of the expression vector that can be used include E. coli-derived plasmids such as pBR322, pBR325, pUC12 or pUC13; B. subtilis-derived plasmids such as pUB110, pTP5 or pC194; yeast-derived plasmids such as pSH19 or pSH15; bacteriophages such as Aphages; viruses such as adenovirus, adeno-associated virus, lentivirus, vaccinia virus, baculovirus or cytomegalovirus; and modified vectors thereof. In view of the activation of gene expression in vivo, a virus vector, particularly an adeno-associated virus, is preferable.

In the aforementioned expression vector, there are no particular limitations on the promoters for expression of the aforementioned dSaCas9 variant protein or the aforementioned guide RNA, and examples thereof that can be used include promoters for expression in animal cells such as EF1α promoter, SRα promoter, SV40 promoter, LTR promoter, cytomegalovirus (CMV) promoter or HSV-tk promoter, promoters for expression in plant cells such as the 35S promoter of cauliflower mosaic virus (CaMV) or rubber elongation factor (REF) promoter, and promoters for expression in insect cells such as polyhedrin promoter or p10 promoter. The se promoters can be suitably selected according to the aforementioned dSaCas9 variant protein and the aforementioned guide RNA, or the type of cells expressing the aforementioned Cas9 protein and the aforementioned guide RNA.

The aforementioned expression vector may also further have a multi-cloning site, enhancer, splicing signal, polyadenylation signal, selection marker (drug resistant) and promoter thereof, or replication origin and the like.

### <Method for Site-Specifically Modifying Target Double-Stranded Polynucleotide>

### First Embodiment

In one embodiment thereof, the present invention provides a method for site-specifically modifying a target double-stranded polynucleotide, provided with:

a step for mixing and incubating a target double-stranded polynucleotide, a protein and a guide RNA, and

a step for having the aforementioned protein modify the aforementioned target double-stranded polynucleotide at a binding site located upstream of a PAM sequence; wherein,

the aforementioned target double-stranded polynucleotide has a PAM sequence,

the aforementioned protein is a protein shown in the aforementioned <dSaCas9 variant>, and

the aforementioned guide RNA contains a polynucleotide composed of a base sequence complementary to a base sequence located 1 to 20 to 24 bases upstream from the aforementioned PAM sequence in the aforementioned target double-stranded polynucleotide.

In the present embodiment, the target double-stranded polynucleotide is not particular limited as long as it has a PAM sequence.

In the present embodiment, the protein and guide RNA are as described in the aforementioned <dSaCas9 variant>.

The following provides a detailed explanation of the method for site-specifically modifying a target double-stranded polynucleotide.

First, the aforementioned protein and the aforementioned guide RNA are mixed and incubated under mild conditions. Mild conditions are as previously described. The incubation time is preferably 0.5 hr to 1 hr. A complex formed by the aforementioned protein and the aforementioned guide RNA is stable and is able to maintain stability even if allowed to stand for several hours at room temperature.

Next, the aforementioned protein and the aforementioned guide RNA form a complex on the aforementioned target double-stranded polynucleotide. The aforementioned protein recognizes PAM sequences, and binds to the aforementioned target double-stranded polynucleotide at a binding site located upstream of the PAM sequence. Successively, a target double-stranded polynucleotide modified to meet the purpose can be obtained in a region determined by the complementary binding of the aforementioned guide RNA and the aforementioned double-stranded polynucleotide.

In the present description, the “modification” means that the target double-stranded polynucleotide changes structurally or functionally. For example, structural or functional change of the target double-stranded polynucleotide by the addition of a functional protein or a base sequence can be mentioned. By the modification, the function of the target double-stranded polynucleotide can be altered, deleted, enhanced, or suppressed, and a new function can be added.

Since the dSaCas9 variant of the present invention does not have endonuclease activity, the protein can bind to the aforementioned target double-stranded polynucleotide at the binding site located upstream of the PAM sequence bond, but stays there and cannot cleave the polynucleotide. Therefore, for example, when a labeled protein such as fluorescent protein (e.g., GFP) and the like is fused to the protein, the labeled protein can be bound to the target double-stranded polynucleotide via dSaCas9 variant protein-guide RNA. By appropriately selecting a substance to be bound to the dSaCas9 variant, various functions can be imparted to the target double-stranded polynucleotide.

Furthermore, transcriptional regulator protein or domain can be linked to the N-terminal or C-terminal of the dSaCas9 variant protein. Examples of the transcriptional regulator or domain thereof include transcriptional activator or domain thereof (e.g., VP64, VP160, NF-KB p65), transcriptional silencer or domain thereof (e.g., hetero chromatin protein 1(HP1)), and transcriptional inhibitor or domain thereof (e.g., Kruppel-associated box (KRAB), ERF repressor domain (ERD), mSin3A interaction domain (STD)).

It is also possible to link an enzyme that modifies the methylation state of DNA (e.g., DNA methyltransferase (DNMT), TET)), or an enzyme that modifies a histone subunit (e.g., histone acetyltransferase (HAT), histone deacetylase (HDAC), histone methyltransferase, histone demethylase).

### Second Embodiment

In the present embodiment, an expression step may be further provided prior to the incubation step in which the protein described in the previous section on GdSaCas9 variant> and guide RNA are expressed using the previously described CRISPR-Cas vector system.

In the expression step of the present embodiment, dSaCas9 variant protein and guide RNA are first expressed using the aforementioned CRISPR-Cas vector system. A specific expression method includes transforming a host using an expression vector containing a gene that encodes dSaCas9 variant protein and an expression vector containing guide RNA, respectively (or expression vector simultaneously containing gene encoding dSaCas9 variant protein and guide RNA). The n, the host is cultured to express the dSaCas9 variant protein and guide RNA. Conditions such as medium composition, culture temperature, duration of culturing or addition of inducing agents can be determined by a person with ordinary skill in the art in accordance with known methods so that the transformant grows and the aforementioned protein is efficiently produced. In addition, in the case of having incorporated a selection marker in the form of an antibiotic resistance gene in the expression vector, the transformant can be selected by adding antibiotic to the medium. The n, the dSaCas9 variant protein and guide RNA are obtained by purifying the dSaCas9 variant protein and guide RNA expressed by the host according to a suitable method.

### <Method for Site-Specifically Modifying Target Double-Stranded Polynucleotide in Cells>

In one embodiment thereof, the present invention provides a method for site-specifically modifying a target double-stranded polynucleotide in cells, provided with:

a step for introducing the previously described CRISPR-Cas vector system into a cell and expressing protein described in the previous section on <dSaCas9 variant> and guide RNA, a step for having the aforementioned protein bind with the aforementioned target double-stranded polynucleotide at a binding site located upstream of a PAM sequence, and a step for obtaining a modified target double-stranded polynucleotide in a region determined by complementary binding between the aforementioned guide RNA and the aforementioned target double-stranded polynucleotide; wherein

the aforementioned guide RNA contains a polynucleotide composed of a base sequence complementary to a base sequence located 1 to 20 to 24 bases upstream from the aforementioned PAM sequence in the aforementioned target double-stranded polynucleotide.

In the expression step of the present embodiment, first, dSaCas9 variant protein and guide RNA are expressed in a cell using the aforementioned CRISPR-Cas vector system.

Examples of organisms serving as the origin of the cells targeted for application of the method of the present embodiment include prokaryote, yeast, animal, plant, insect and the like. The re are no particular limitations on the aforementioned animals, and examples thereof include, but are not limited to, human, monkey, dog, cat, rabbit, swine, bovine, mouse, rat and the like. In addition, the type of organism serving as the source of the cells can be arbitrarily selected according to the desired type or objective of the target double-stranded polynucleotide.

Examples of animal-derived cells targeted for application of the method of the present embodiment include, but are not limited to, germ cells (such as sperm or ova), somatic cells composing the body, stem cells, progenitor cells, cancer cells isolated from the body, cells isolated from the body that are stably maintained outside the body as a result of having become immortalized (cell line), and cells isolated from the body for which the nuclei have been artificially replaced.

Examples of somatic cells composing the body include, but are not limited to, cells harvested from arbitrary tissue such as the skin, kidneys, spleen, adrenals, liver, lungs, ovaries, pancreas, uterus, stomach, small intestine, large intestine, urinary bladder, prostate gland, testes, thymus, muscle, connective tissue, bone, cartilage, vascular tissue, blood, heart, eyes, brain or neural tissue. Specific examples of somatic cells include, but are not limited to, fibroblasts, bone marrow cells, immunocyte s (e.g., B lymphocytes, T lymphocytes, neutrophils, macrophages or monocytes etc.), erythrocytes, platelets, osteocytes, bone marrow cells, pericytes, dendritic cells, keratinocytes, adipocytes, mesenchymal cells, epithelial cells, epidermal cells, endothelial cells, intravascular endothelial cells, lymphatic endothelial cells, hepatocytes, pancreatic islet cells (e.g., a cells, β cells, δ cells, ϵ cells or PP cells etc.), chondrocytes, cumulus cells, glia cells, nerve cells (neurons), oligodendrocytes, microglia cells, astrocytes, cardiomyocytes, esophageal cells, muscle cells (e.g., smooth muscle cells or skeletal muscle cells etc.), melanocytes and mononuclear cells, and the like.

Stem cells refer to cells having both the ability to self-replicate as well as the ability to differentiate into a plurality of other cell lines. Examples of stem cells include, but are not limited to, embryonic stem cells (ES cells), embryonic tumor cells, embryonic germ stem cells, induced pluripotent stem cells (iPS cells), neural stem cells, hematopoietic stem cells, mesenchymal stem cells, hepatic stem cells, pancreatic stem cells, muscle stem cells, germ stem cells, intestinal stem cells, cancer stem cells and hair follicle stem cells, and the like.

Cancer cells are cells derived from somatic cells that have acquired reproductive integrity. Examples of the origins of cancer cells include, but are not limited to, breast cancer (e.g., invasive ductal carcinoma, ductal carcinoma in situ, inflammatory breast cancer etc.), prostate cancer (e.g., hormone-dependent prostate cancer or non-hormone dependent prostate cancer etc.), pancreatic cancer (e.g., pancreatic ductal carcinoma etc.), stomach cancer (e.g., papillary adenocarcinoma, mucinous carcinoma, adenosquamous carcinoma etc.), lung cancer (e.g., non-small cell lung cancer, small cell lung cancer, malignant mesothelioma etc.), colorectal cancer (e.g., gastrointestinal stromal tumor etc.), rectal cancer (e.g., gastrointestinal stromal tumor etc.), colorectal cancer (e.g., familial colorectal cancer, hereditary non-polyposis colorectal cancer, gastrointestinal stromal tumor etc.), small intestinal cancer (e.g., non-Hodgkin's lymphoma, gastrointestinal stromal tumor etc.), esophageal cancer, duodenal cancer, cancer of the tongue, pharyngeal cancer (e.g., nasopharyngeal carcinoma, oropharyngeal carcinoma, hypopharyngeal carcinoma etc.), head and neck cancer, salivary gland cancer, brain tumor (e.g., pineal astrocytoma, pilocytic astrocytoma, diffuse astrocytoma, anaplastic astrocytoma etc.), schwannoma, liver cancer (e.g., primary liver cancer, extrahepatic bile duct cancer etc.), kidney cancer (e.g., renal cell carcinoma, transitional cell carcinoma of the renal pelvis and ureter etc.), gall bladder cancer, bile duct cancer, pancreatic cancer, endometrial carcinoma, cervical cancer, ovarian cancer (e.g., epithelial ovarian cancer, extragonadal germ cell tumor, ovarian germ cell tumor, ovarian low malignant potential tumor etc.), bladder cancer, urethral cancer, skin cancer (e.g., intraocular (ocular) melanoma, Merkel cell carcinoma etc.), Hemangioma, malignant lymphoma (e.g., reticulum cell sarcoma, lymphosarcoma, Hodgkin's etc.), melanoma (malignant melanoma), thyroid cancer (e.g., medullary thyroid cancer etc.), parathyroid cancer, nasal cancer, paranasal cancer, bone tumor (e.g., osteosarcoma, Ewing's tumor, uterine sarcoma, soft tissue sarcoma etc.), metastatic medulloblastoma, vascular fibroma, protuberant dermatofibrosarcoma, retinal sarcoma, penile cancer, testicular cancer, pediatric solid tumor (e.g., Wilms tumor or pediatric kidney tumor etc.), Kaposi's sarcoma, AIDS-induced Kaposi's sarcoma, maxillary sinus tumor, fibrous histiocytoma, leiomyosarcoma, rhabdomyosarcoma, chronic myeloproliferative disease and leukemia (e.g., acute myeloid leukemia, acute lymphoblastic leukemia etc.).

Cell lines refer to cells that have acquired reproductive integrity through artificial manipulation ex vivo. Examples of cell lines include, but are not limited to, HCT116, Huh7, HEK293 (human embryonic kidney cells), HeLa (human cervical cancer cell line), HepG2 (human liver cancer cell line), UT7/TPO (human leukemia cell line), CHO (Chinese hamster ovary cell line), MDCK, MDBK, BHK, C-33A, HT-29, AE-1, 3D9, NsO/1, Jurkat, NTH3T3, PC12, S2, Sf9, Sf21, High Five and Vero.

Introduction of the CRISPR-Cas vector system into cells can be carried out using a method suitable for the viable cells used, and examples thereof include electroporation method, heat shock method, calcium phosphate method, lipofection method, DEAE dextran method, microinjection method, particle gun method, methods using viruses, and methods using commercially available transfection reagents such as FuGENE (registered trade mark) 6 Transfection Reagent (manufactured by Roche), Lipofectamine 2000 Reagent (manufactured by Invitrogen Corp.), Lipofectamine LTX Reagent (manufactured by Invitrogen Corp.) or Lipofectamine 3000 Reagent (manufactured by Invitrogen Corp.).

The subsequent modification step is the same as the method shown in the aforementioned <Method for Site-Specifically Modifying Target Double-Stranded nucleotide> [First Embodiment].

By the modification of the target double-stranded polynucleotide in this embodiment, cells with modified target double-stranded polynucleotide can be obtained.

While the present invention is explained in more detail in the following by referring to Examples, they do not limit the scope of the present invention.

### EXAMPLE

### Example 1: Evaluation of DNA Binding Affinity of dSaCas9 Variant

### (Method)

### 1. Cloning

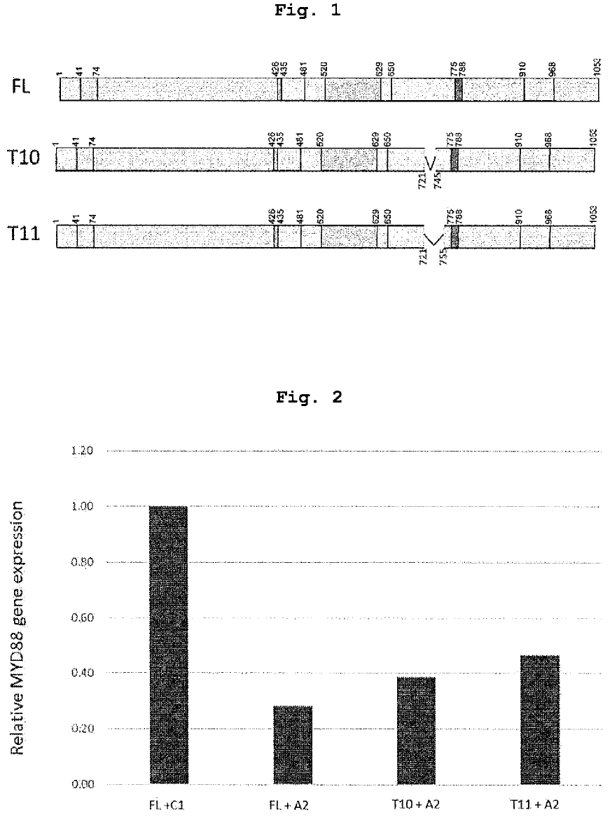

Using NEB Q5 Site-Directed Mutagenesis Kit, a predetermined deletion region was made in dSaCas9 gene, a gene encoding a linker was introduced, and a KRAB gene as a transcriptional regulator was fused to produce various dSaCas9s (FIG. 1). The expression suppression activity of these dSaCas9s was examined using MYD88 gene. All gene constructs of dSaCas9 were incorporated into pX601 vectors (F. Ann Ran et al., Nature 2015; 520(7546); pp.186-191). Each vector expresses the following dSaCas9 constructs (FL, T10, T11) and sgRNA.

[dSaCas9 variants]
(SEQ ID NO: 1, SEQ ID NO: 2)
FL; wild-type dSaCas9

T10; deletion variant (SEQ ID NO: 3, SEQ ID NO: 4) having a deletion region (721- to 745-positions) in wild-type dSaCas9 T11; deletion variant (SEQ ID NO: 5, SEQ ID NO: 6) having a deletion region (721- to 755-positions) in wild-type dSaCas9

Both ends of the respective deletion regions in T10 and T11 are linked by a linker peptide.

dSaCas9 construct is expressed as a fusion protein of dSaCas9-KRAB-P2A-sfGFP or dSaCas9-VR-P2A-sfGFP.

[crRNA sequences]
C1;
(SEQ ID NO: 11)
ACGGAGGCUAAGCGUCGCAA
A2;
(SEQ ID NO: 12)
GGAGCCACAGUUCUUCCACGG
A3;
(SEQ ID NO: 13)
CUCUACCCUUGAGGUCUCGAG

The above-mentioned crRNA sequence was fused with the following tracrRNA sequence to form sgRNA to be expressed from the vector.

(SEQ ID NO: 14)
GUUUUAGUACUCUGGAAACAGAAUCUACUAAAACAAGGCAAAAUGCCGU
GUUUAUCACGUCAACUUGUUGGCGAGAUUUUUUU

### 2. Cell Transfection

HEK293FT cells were seeded in a 24-well plate at a density of 75,000 cells per well 24 hr before transfection and cultured in DMEM medium supplemented with 10% FBS, 2 mM fresh L-glutamine, 1 mM sodium pyruvate and non-essential amino acid. Each plasmid (500 ng) was transfected into cells according to the manual and using 1.5 μl of Lipofectamine 2000 (Life technologies). For the gene expression analysis, the cells were recovered at 48-72 hr after transfection, dissolved in RLT buffer (Qiagen), and the total RNA was extracted using RNeasy kit (Qiagen).

### 3. Gene Expression Analysis

For the Tagman analysis, cDNA was prepared from 1.5 μg of the total RNA by using 20 μl volume TagMan™ High-Capacity RNA-to-cDNA Kit (Applied Biosystems). The prepared cDNA was diluted 20-fold and 6.33 μl was used for each Tagman reaction. The Tagman primers and probes for the MYD88 gene and HPRT gene were obtained from Applied Biosystems. In Roche LightCycler 96 or LightCycler 480, the Tagman reaction was run using Taqman gene expression master mix (ThermoFisher), and the analysis was performed using LightCycler 96 analysis software. Tagman probe product IDs:

### MYD88: Hs01573837_g1 (FAM)

### HPRT: Hs99999909_m1 (FAM, VIC)

### Tagman QPCR Condition:

### Step 1; 95° C. 10 min

### Step 2; 95° C. 15 sec

### Step 3; 60° C. 30 sec

Repeat Step 2 and 3; 40 times

### (Results)

HEK293FT cells were transfected with a plasmid vector expressing pX601-dSaCas9 (FL, T10 or T11)-KRAB-P2A-sfGFP and sgRNA-A2. The cells transfected with a vector expressing sgRNA-C1 instead of sgRNA-A2 were used as a negative control. The transfected cells were harvested 3 days later and RNA was isolated. The expression of the MYD88 gene was analyzed by Tagman assay and standardized at the expression level of the HPRT gene. The results of the relative expression level of the MYD88 gene are shown in FIG. 2.

When compared with the control, the MYD88 gene expression level in the dSaCas9 variant of the present invention was as low as that of the wild-type dSaCas9. From the results, it was shown that the binding ability to the guide RNA, and further, the DNA binding affinity, were maintained even though the dSaCas9 variant of the present invention has a deletion region and is smaller than the full-length dSaCas9.

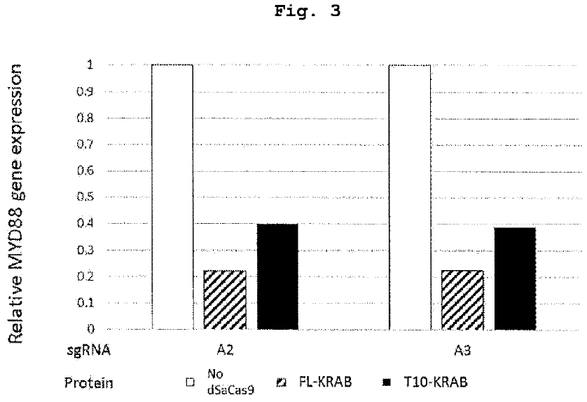

The binding affinity of T10 which showed particularly high DNA binding affinity in the above-mentioned results was confirmed using a different guide RNA. HEK293FT cells were transfected with a plasmid vector expressing pX601-dSaCas9 (FL or T10)-KRAB-P2A-sfGFP and sgRNA-A2 or sgRNA-A3. The cells transfected with a vector not expressing dSaCas9 variant were used as a negative control. The transfected cells were harvested 3 days later and RNA was isolated. The expression of the MYD88 gene was analyzed by Tagman assay and standardized at the expression level of the HPRT gene. The results of the relative expression level of the MYD88 gene are shown in FIG. 3. Even when sgRNA-A3 was used, an effect similar to that of sgRNA-A2 was confirmed.

When compared with the control, the MYD88 gene expression level in the dSaCas9 variant of the present invention was as low as that of the wild-type dSaCas9. From the results, it was shown that the binding ability to the guide RNA, and further, the DNA binding affinity, were maintained even though the dSaCas9 variant of the present invention has a deletion region and is smaller than the full-length dSaCas9.

### INDUSTRIAL APPLICABILITY

According to the present invention, a dSaCas9 protein that is miniaturized while maintaining a DNA binding ability can be obtained. Use of the miniaturized dSaCas9 protein makes it possible to mount many genes into vectors, and thus provides various genome editing techniques.

This application is based on a provisional patent application No. 62/749,855 filed in the US (filing date: Oct. 24, 2018), the contents of which are incorporated in full herein.

1. A protein having a binding ability to guide RNA and consisting of a sequence comprising an amino acid sequence wherein a continuous deletion region is present between the 721-position and the 755-position in the amino acid sequence shown in SEQ ID NO: 2,
wherein amino acids adjacent to each of the deletion region are linked by a linker consisting of 3 to 10 amino acid residues.

2. The protein according to claim 1, wherein the linker is a 5-9 amino acid length linker composed of glycine (G) and serine (S).

3. The protein according to claim 2, wherein the linker is selected from the following:
—SGGGS—
—GGSGGS—
—SGSGSGSG1'
—SGSGSGSGS—.

4. The protein according to claim 1, wherein the deletion region is a region of the 721-position to the 745-position.

5. The protein according to claim 4, wherein the protein is shown in SEQ ID NO: 4.

6. The protein according to claim 1, wherein the deletion region is a region of the 721-position to the 755-position.

7. The protein according to claim 6, wherein the protein is shown in SEQ ID NO: 6.

8. The protein according to claim 1, wherein glutamic acid (E) at the 45-position and/or the 163-position are/is substituted with other amino acid(s).

9. The protein according to claim 8, wherein said other amino acid is a basic amino acid.

10. The protein according to claim 9, wherein the basic amino acid is lysine (K).

11. The protein according to claim 1, having identity of 80% or more at a site other than the mutated and/or deleted positions in the SEQ ID NO: 2.

12. The protein according to claim 1, wherein one to several amino acids are substituted, deleted, inserted and/or added at a site other than the mutated and/or deleted positions in the SEQ ID NO: 2.

13. The protein according to claim 1, wherein a transcriptional regulator protein or domain is linked.

14. The protein according to claim 13, wherein the transcriptional regulator is a transcriptional activator.

15. The protein according to claim 13, wherein the transcriptional regulator is a transcriptional silencer or a transcriptional inhibitor.

16. A nucleic acid encoding the protein according to claim 1.

17. A protein-RNA complex provided with the protein according to claim 1 and a guide RNA comprising a polynucleotide composed of a base sequence complementary to a base sequence located 1 to 20 to 24 bases upstream from a proto-spacer adjacent motif (PAM) sequence in a target double-stranded polynucleotide.

18. A method for site-specifically modifying a target double-stranded polynucleotide, including
a step of mixing and incubating a target double-stranded polynucleotide, a protein and a guide RNA, and
a step of having the aforementioned protein modify the aforementioned target double-stranded polynucleotide at a binding site located upstream of a PAM sequence; wherein,
the aforementioned protein is the protein according to claim 1, and
the aforementioned guide RNA contains a polynucleotide composed of a base sequence complementary to a base sequence located 1 to 20 to 24 bases upstream from the aforementioned PAM sequence in the aforementioned target double-stranded polynucleotide.

19. A method for increasing expression of a target gene in a cell, comprising expressing the protein according to claim 14 and one or plural guide RNAs for the aforementioned target gene in the aforementioned cell.

20. A method for decreasing expression of a target gene in a cell, comprising expressing the protein according to claim 15 and one or plural guide RNAs for the aforementioned target gene in the aforementioned cell.

21. The method according to claim 19, wherein the cell is a eukaryotic cell, a yeast cell, a plant cell or an animal cell.

22. The method according to claim 20, wherein the cell is a eukaryotic cell, a yeast cell, a plant cell or an animal cell.
